(19) United States
(12) Patent Application Publication
(10) Pub. No.: US 2012/0142039 A1
(43) Pub. Date: Jun. 7, 2012

(54) METHOD FOR PREDICTING GLYCAEMIC RESPONSE AND USE THEREOF

(75) Inventors: Robert Havenaar, Zeist (NL); Mans Minekus, Werkhoven (NL); Susann Christiene Bellmann, Wageningen (NL)

(73) Assignee: Nederlandse Organisatie voor toegepast-naruurwetenschappelijk onderzoek TNO, Delft (NL)

(21) Appl. No.: 13/377,269

(22) PCT Filed: Jun. 11, 2010
(86) PCT No.: PCT/NL2010/050357

§ 371 Date: Feb. 21, 2012

(30) Foreign Application Priority Data
Jun. 11, 2009 (EP) 09162517.8

(51) Int. Cl. C12Q 1/40; C12Q 1/37; C12Q 1/34

(52) U.S. Cl. 435/22; 435/18; 435/23

(57) ABSTRACT

The present invention relates to an in vitro method for predicting the in vivo glycaemic response to carbohydrates in a food. Provided is a method wherein a food is digested in vitro in more than one compartment. Also provided is the use of such a method for determining the suitability of a food for a subject who would benefit from a normalization of blood glucose levels.

### INTRODUCTION

The present invention relates to an in vitro method for predicting the in vivo glycaemic response to carbohydrates in a food. Provided is a method wherein a food is digested in vitro in more than one compartment. Also provided is the use of such a method for determining the suitability of a food for a subject who would benefit from a normalization of blood glucose levels.

### BACKGROUND OF THE INVENTION

The effects of carbohydrates in a food on in vivo blood glucose levels can be measured by determining the food's glycaemic response or glycaemic index (also glycemic index), abbreviated as GI (Jenkins et al., American Journal of Clinical Nutrition 1981 vol. 34: 362-366). First, an amount of the food to be tested is determined so that the amount comprises a fixed portion of carbohydrates, usually 50 grams. Following the in vivo ingestion and digestion of this pre-determined amount of test food over time, a two hour blood glucose response curve can be determined. The area under the curve (AUC) is then divided by the AUC of a reference food and multiplied by 100. The outcome of this calculation denotes the GI of the food. The current validated methods use glucose as the reference food, giving it a GI value of 100 by definition. The average GI value for a test food is calculated from data collected in 10 human subjects (Brouns et al., Nutrition Research Reviews 2005 vol. 18: 145-171).

Carbohydrates with a high GI (70 and above) are carbohydrates that break down rapidly during digestion, causing glucose to be released into the bloodstream quickly and thus eliciting a large glycaemic response. Examples of foods containing such carbohydrates are cornflakes, baked potato and white bread. Carbohydrates with a low GI (55 or less) are less digestible carbohydrates or carbohydrates that break down slowly, resulting in a lower or gradual release of glucose into the bloodstream. Examples of foods containing such carbohydrates are grainy breads such as pumpernickel, most fruits and vegetables and brown rice. Whole wheat products, table sugar, beer and most species of white rice are examples of foods containing carbohydrates with a medium GI (56-69).

Low glycaemic diets have been reported to improve both glucose and lipid levels in people with diabetes mellitus (type 1 and 2), to result in benefits in weight control because they help control appetite and hunger and to reduce insulin levels and insulin resistance.

Furthermore, several lines of scientific evidence have shown that individuals who followed a low GI diet over many years were at a significantly lower risk than others for developing both type 2 diabetes mellitus and coronary heart disease. High blood glucose levels or repeated glycaemic “spikes” following a meal may promote these diseases by increasing oxidative damage to the vasculature, and also by the direct increase in insulin levels (Temelkova-Kurktschiev et al., Diabetes Care 2000 vol. 23(12): 1830-1834). In the past, postprandial hyperglycemia has been considered a risk factor mainly associated with diabetes. However, more recent research shows that it also presents an increased risk for atherosclerosis in the non-diabetic population (Balkau et al., Diabetes Care 1998 vol. 21(3): 360-367).

Thus, foods with a low GI (i.e., foods that elicit a small glycaemic response) are considered to provide significant health benefits in general, especially for subjects suffering from or having increased risk for developing syndromes such as glucose intolerance, insulin resistance, diabetes mellitus, syndrome X or metabolic syndrome, cardiovascular disease and obesity. It is therefore often recommended that consumers modify their overall diets such that the relative amount of low GI foods is increased, at the expense of high GI foods.

Foods with a high GI (i.e., food that elicits a large glycaemic response) may however also provide benefits, depending on their intended use. For example, as such foods cause a more rapid rise in blood glucose levels they are suitable for a person in need of energy recovery after endurance exercise or for a person suffering from diabetes mellitus who is experiencing hypoglycemia.

As glycaemic response values can be used to assist consumers and instances such as health care providers in selecting foods and possibly reducing the risk of certain diseases, methods to accurately predict the in vivo glycaemic response values or GI of carbohydrates in a food are needed.

The “gold standard” for calculating a food's in vivo GI is to measure the changes in blood glucose levels that occur over time after consumption and digestion of a fixed amount of that food. The obtained in vivo glucose response is then compared to the in vivo glucose response of a reference food with a known GI, usually glucose. As mentioned above, the average GI value for a test food is typically calculated from data collected in 10 human subjects.

However, this in vivo method suffers from considerable drawbacks. Not only does it require participation of human subjects, it also requires overnight fasting and frequent invasive blood sampling. Also, the method comprises not one but two tests as the glucose response to the reference food has to be measured as well. Thus, it may be considered unethical.

The method is also rather time consuming and costly. Most importantly, compliance of the participants to the test protocol is difficult to guarantee, casting doubts about the reliability of the obtained results. Alternative methods are therefore needed that avoid participation of human subjects and require less time and money, without loss of accuracy in calculating glycaemic responses as compared to in vivo conditions.

Although there has been considerable interest in developing in vitro protocols only a very few have actually been established, of which the best known is the method by Englyst et al. The initial method of Englyst et al. involved the measurement of glucose, released from a crushed test food during incubation at 37° C. with HCl and a mixture of digestive enzymes, using a calorimetric assay to determine the final glucose level (Englyst et al., British Journal of Nutrition 1996 75: 327-337). It was modified a few years later by replacing the calorimetric assay with an HPLC assay to improve reproducibility (Englyst et al., American Journal of Clinical Nutrition 1999 69: 448-454), and it has remained unchanged ever since. The final in vitro method by Englyst et al. using the HPLC endpoint is hereinafter referred to as the “Englyst method”.

The Englyst method also suffers from drawbacks. The conditions under which the tested food is digested are not comparable to in vivo digesting conditions. For example, the first digesting step involves crushing or mincing a food sample to an “as eaten” state without any enzymatic pre-digestion of the food, such as by amylase. Also, after addition of HCl and pepsin the minced food mix is only buffered back to pH 5.2 instead of to at least neutral value (pH=7-8) as would occur in an in vivo intestinal tract. Enzymes for digesting the carbohydrates in the food sample are not added until after the pH has been buffered.

Furthermore, the Englyst method does not involve different digestive compartments. All the reacting substances are mixed together in one and the same tube. Thus, the typical conditions that are met by the food in the various digestive compartments in vivo are not provided. Moreover, the Englyst method does not account for peristaltic movement of the food.

Also, only two samples are taken during the whole incubation procedure. The first sample, G₂₀, is taken exactly 20 minutes after addition of the enzyme mixture and is used to determine the rapidly available glucose (RAG). The second sample, G₁₂₀, is taken exactly 120 minutes after addition of the enzyme mixture. A portion of the G₁₂₀ sample is used to determine the slowly available glucose (SAG). The remainder of said sample is then used to determine the total glucose content in the sample. An interpretation that would better resemble the whole picture of the natural in vivo human glucose response is not provided.

Several authors have criticized the Englyst method, for example for providing little predictive value (Garsetti et al., J Am Coll Nutr 2005 24: 441-447) and for incorrect classification of commercial available foods (Brand-Miller et al., European Journal of Clinical Nutrition 2004 58: 700-701). These authors strongly advise against the use of any in vitro method for producing GI values for food labeling purposes.

The difficulties of developing a reliable in vitro method to predict the in vivo glycaemic response to a food are demonstrated by the simple fact that until now only one other example of such a method has been described.

WO 2009/006155, published in January 2009, describes an in vitro method for the prediction of the GI of food products. Initially, the fat and protein contents of a food product are determined, whereafter it is ground to a homogenous state. The food product is then digested with a mixture of digestive enzymes for a fixed period of time to provide a digested sample. After treatment of the digested sample to stop enzymatic reactions, amounts of glucose and at least two other monosaccharides (fructose, galactose, lactose, sucrose and/or maltitol) are determined. The in vivo GI of the food product is then predicted using a predictive equation or method derived from multivariate analysis with calibration data. The calibration data are obtained from calibration samples with known in vivo GI values.

At first glance this method looks promising. However, the in vitro method according to WO 2009/006155 also does not mimic the physiological conditions that occur when a food is digested in vivo. It seems to be predominantly based on the principles of the Englyst method with only few procedural adaptations. Consequently, it suffers from the same drawbacks as the Englyst method does.

For example, this in vitro method requires the food product to be ground to an “essentially homogeneous and finely divided state” which is defined as a degree of grinding equivalent to grinding the food product at temperatures sufficiently low such that the food is a brittle solid. According to WO 2009/006155 such temperatures are generally lower than about −40° C., preferably below about −78° C., and more preferably at about temperatures obtainable using liquid nitrogen (−196° C.). Such conditions are not comparable to in vivo conditions, e.g. when food is chewed in a mouth having a temperature of around body temperature and wherein said food is mixed with salivary fluid that contains digestive enzymes. Also, the need for such conditions when processing a solid food renders the method time-consuming and laborious. As such steps require additional equipment such as cooling equipment, liquid nitrogen and special grinding mills, costs are added as well. Thus, the in vitro method according to WO 2009/006155 seems much less suitable for determining the glycaemic index of solid foods than for determining the glycaemic index of non- or semi-solid foods, where no grinding is involved.

Furthermore, this in vitro method comprises a digestive incubation step that is very similar to the incubation step according to the Englyst method. All mixing and digestive steps take place in one vial, and small glass balls are used to mix the ground food with the digestive substances. Also similar to the Englyst method, after addition of HCl and pepsin the ground food is only buffered back to pH at around 5 instead of to at least neutral value (pH=7-8) as would occur in an in vivo intestinal tract, and enzymes for digesting the carbohydrates in the food sample are not added until after the pH has been buffered. Thus, also the in vitro method according to WO 2009/006155 does not resemble the physiological environment and involves only one digestive compartment. It again does not provide the typical peristaltic movement of the food from one digestive compartment to another that would occur during in vivo digestion of a food.

After exactly 20 minutes (±10 seconds) of digestive incubation, a sample is taken for determination of the glycaemic index of the food product. This sampling procedure is comparable to the procedure for measurement of rapidly available glucose (RAG) according to the Englyst method that also uses a food sample taken after exactly 20 minutes. It only differs in that also data concerning the fat and protein contents of the food product as well as the content of carbohydrates other than glucose are used to determine the glycaemic index of the carbohydrates in the food product. Again, an interpretation that would better resemble the whole picture of the natural in vivo human glucose response is not provided.

Thus, the need for an in vitro method for predicting the in vivo glycaemic response to carbohydrates in a food that is accurate and that resembles in vivo physiological conditions remains.

### SUMMARY OF THE INVENTION

The current invention provides such a method. Provided is a method for predicting the in vivo glycaemic response to carbohydrates in a food wherein the food is digested in vitro, characterized in that the food is digested in more than one compartment. In one embodiment, said food is digested in two compartments. Also provided is such a method, comprising digesting said food with at least one digestive enzyme selected from the group of amylase, lipase, pepsin, trypsin, chymotrypsin, pancreatin and brush border enzymes. Further provided is such a method, comprising digesting said food with gastric acid. Even further provided is a method for predicting the in vivo glycaemic response to the carbohydrates in a food, comprising applying pressure to said food. Preferably, said pressure is applied in a peristaltic manner. More preferably, said pressure is mechanically induced.

Also provided is a method for predicting the in vivo glycaemic response to carbohydrates in a food, comprising transferring said food from one compartment to another in a peristaltic motion. In one embodiment, said peristaltic motion is mechanically induced.

Further provided is a method according to the invention, comprising collecting at least one sample from said food before, during, and/or after digestion. Preferably, said method comprises digesting at least one carbohydrate in said at least one sample collected from said food. Also preferred is a method comprising measuring the amount of at least one saccharide selected from the group consisting of glucose, fructose, galactose, lactose and sucrose in said at least one sample collected from said food. Further preferred, said method comprises calculating the in vitro glucose or fructose response or both to said food.

Also provided is the use of a method according to the invention for determining the suitability of a food for a subject who would benefit from a normalization of blood glucose levels. Preferably, said subject is suffering from or having increased risk for developing at least one syndrome selected from the group of glucose intolerance, insulin resistance, diabetes mellitus, syndrome X, cardiovascular disease and obesity.

It should be understood that this invention is not limited to the embodiments disclosed in this summary, but it is intended to cover modifications that are within the spirit and scope of the invention, as defined in the claims.

### LEGENDS TO THE FIGURES

FIG. 1: Calibration data representing the predicted versus observed in vivo glycaemic responses to 50 grams of pumpernickel whole kernel rye bread in young volunteers (Juntunen et al., 2002).

FIG. 2: Predicted plasma glucose curve (closed dots, n=2) in comparison to the actual in vivo plasma glucose curve (open triangles, n=10, mean±SEM) after the consumption of Durum wheat semolina pasta. Dotted lines indicate±5% deviation from the actual in vivo curve.

FIG. 3: Predicted plasma glucose curve (closed dots, n=2) in comparison to the in vivo plasma glucose curve (open triangles, n=9, mean±SEM) after the consumption of Parboiled pearled wheat. Dotted lines indicate±5% deviation from the in vivo curve.

FIG. 4: Predicted plasma glucose curve (closed dots, n=2) in comparison to the in vivo plasma glucose curve (open triangles, n=6, mean±SEM) after the consumption of Kellogg's Special K. Dotted lines indicate±5% deviation from the in vivo curve.

FIG. 5: Predicted plasma glucose curve (closed dots, n=2) in comparison to the in vivo plasma glucose curve (open triangles, n=10, mean±SEM) after the consumption of Nutrient formula A. Dotted lines indicate ±5% deviation from the in vivo curve.

### DETAILED DESCRIPTION OF THE INVENTION

### Definitions

The term “digestion” as used herein is defined as any process or method by which a food is prepared for absorption into the blood. This includes in vivo and in vitro degradation of a food. It comprises any process or method for preparing a food for consumption, such as cooking, baking or frying, any method or process for crushing a food such as chewing or grinding, any method or process for degrading the macromolecules in a food such as enzymatic digestion or acidic hydrolysis, any method or process for mixing a food with digestive compounds, such as peristalsis or application of mechanical pressure and any method or process for transporting a food through a gastrointestinal tract or from one compartment to another, such as peristaltic movement.

As used herein, “carbohydrate” means any compound of general structure (CH₂O)n, wherein n is a positive integer and n≧3, that can be digested by a human, animal, bacterium or fungus, including yeast. This includes, but is not limited to, all mono-, di-, oligo- and polysaccharides.

The term “monosaccharides” as used herein means any compound of general structure (CH₂O)n, wherein n is a positive integer and 3≦n≦9, that can be produced in and possibly absorbed from the gastrointestinal tract of a human or an animal. This includes, but is not limited to, glucose, fructose, galactose, whether or not hydrogenated or otherwise modified, including but not limiting to sorbitol, mannitol and xylitol.

The term “digestive enzyme” as used herein means any enzyme, purified as well as in (natural) mixtures, that breaks down or modifies di-, oligo- or polymeric macromolecules into their smaller building blocks or other molecule structures and that is of human, animal, bacterial or fungal origin, or produced by biotechnological processes. It includes any enzyme that breaks down or modifies carbohydrates such as, but not limited to, ptyalin, α-amylase or β-amylase, isomaltase, maltase, sucrase and lactase. It also includes any enzyme that breaks down or modifies proteins or peptides such as, but not limited to, pepsin, trypsin, chymotrypsin, peptidase, carboxypeptidase, peptase, elastase, gelatinase and nuclease. Furthermore, it includes any enzyme that breaks down or modifies fat such as, but not limited to, lipase, steapsin and pancreatin.

As used herein, the term “brush border enzymes” is defined as a mixture comprising at least one of the digestive enzymes that are located in the layer of the brush border (microvilli) on intestinal epithelial cells of humans and animals such as, but not limited to, aminopeptidases, glucoamylase (also known in the art as maltase), sucrase (also known in the art as sucrase-isomaltase) and lactase (also known as galactosidase).

The term “compartment” as used herein is defined as any space that comprises at least one wall and is suitable for containing a food and digestive enzymes. Thus, the term includes any such space wherein the at least one wall is made from a rigid material such as a glass or a metal, and any such space wherein the at least one wall is made from a non-rigid material such as rubber. It also includes any such space further comprising an inner wall or structure made from a non-rigid material, such as a plastic layer or a rubber hose. It further includes any such space being open at at least one end, and any such space comprising one or more connecting passages. Finally, the term includes any such space comprising two or more subspaces that each comprise at least one wall, are suitable for containing a food and digestive enzymes and that are connected to each other by means of at least one connecting passage. Non-limitative examples of a compartment according to the definition are a glass cup, a metal beaker, a plastic vial, a rubber tube, a rubber sac and a tube that comprises an inner hose made of rubber and that is connected to another compartment.

As used herein, “peristalsis” means any series of successive muscle contractions passing along the walls of a hollow muscular structure (such as the oesophagus, stomach or intestines), applying pressure to the contents of that structure and forcing them onward or backward.

The term “peristaltic movement” as used herein is defined as any in vitro movement of the content in a compartment, including a food, that is induced by any process resembling peristalsis. This includes any in vitro movement of the content and/or a food within a compartment and any in vitro movement from a food from one compartment to another.

### Description

The inventors surprisingly found that the accuracy of predicting the in vivo glycaemic response to a food is increased when said food is digested in more than one compartment. Provided is a method for predicting the in vivo glycaemic response to carbohydrates in a food wherein the food is digested in vitro, characterized in that the food is digested in more than one compartment. The method can be used to predict the in vivo glycaemic response to any type of food. Such food may be a solid food, a semi-solid food or a non-solid food. A “food” as used herein can be a single food component, including a liquid or a solid food, and it may be a food matrix made up of a mixture of single food components up to a complete meal.

The number of compartments may vary depending on the type of food to be tested. For example, digestion of a liquid food may require fewer compartments than digestion of a solid food. In one embodiment, the invention provides a method for predicting the in vivo glycaemic response to carbohydrates in a food wherein the food is digested in vitro, characterized in that the food is digested in two compartments.

The number of compartments may also depend on the type of digestion that is required. It may for instance be required that the digestion resembles the digestion by humans or animals. Animal digestion can be of different types, such as digestion by a carnivore, an omnivore or a herbivore. For example, a digestion may resemble human, mammalian or avian digestion. More specifically a digestion may resemble human, digestion or digestion of an animal selected from, but not limited to, the group consisting of pig, horse, dog, cat, mouse, rat, cow, sheep, chicken and ostrich. Therefore the method may comprise digesting a food in three or more compartments.

It may thus be required that the digestion resembles the typical digestion by a ruminant such as a cow or a deer. Thus, in yet another embodiment the method comprises digesting a food in more than three compartments. For example, a food is digested with amylase in one compartment, digested with gastric acid in another compartment, digested with trypsin and lipase in a further compartment and then fermented with bacteria in yet a further compartment. Thus, a skilled person will appreciate that the appropriate number of compartments for digesting a food can be readily determined, depending on the type of food and the type of digestion.

It may be clear from the above that it is preferred that the conditions of an in vitro method for predicting the glycaemic response to a food mimic the in vivo physiological conditions wherever possible. For example, each digestion step of the food should preferably be conducted at a temperature that reflects the average body temperature, i.e. a temperature between 35° C. and 39° C., preferably around 37° C. Thus, not only the temperature of the food to be digested, but also the temperature of the digestion compartments and of the solutions that are added at each compartment should preferably deviate as less as possible from the average body temperature. However, it will be well understood by a person skilled in the art that exceptions may occur. For example, a food may be cooked, baked or prepared otherwise according to instructions before digestion and thus may have a temperature that is above or below body temperature. Nevertheless, it should be avoided to digest food having a temperature of more than about 70° C. or lower than about −20° C.

A food is typically digested by at least one digestive enzyme. A person skilled in the art will appreciate which enzymes are best suited for the in vitro methods according to the invention to digest a certain type of food. For example, enzymes such as salivary and pancreatic amylase, and brush border enzymes such as sucrase and lactase, are best suited for digesting the carbohydrates in a food. Other enzymes such as pepsin, trypsin, chymotrypsin, and lipase may also be used, to free the carbohydrates from the food matrix. To reflect in vivo physiological conditions such digestive enzymes are preferably selected from enzymes of human or animal origin, such as amylase that is found in human and animal salivary fluid.

Therefore, in one aspect the invention provides a method for predicting the in vivo glycaemic response to carbohydrates in a food wherein the food is digested in vitro, characterized in that the food is digested in more than one compartment, comprising digesting said food with at least one digestive enzyme selected from the group of amylase, lipase, pepsin, trypsin, chymotrypsin, pancreatin and brush border enzymes. For example, a food is digested with amylase and pepsin in one compartment and digested with lipase and pancreatin in another compartment. The method may also comprise digesting a food with amylase in one compartment, digesting said food with pepsin in another compartment and digesting said food with pancreatin in yet another compartment.

It will be clear that the digestion of a food can be supported by substances other than digestive enzymes, such as gastric acid and bile. Therefore, in another embodiment the method according to the invention comprises digesting a food with gastric acid. For example, a food is digested with amylase in one compartment and then digested with gastric acid in another compartment. Also other substances are secreted in the digestive tract. For example, potassium chloride and sodium chloride are secreted during in vivo digestion in the stomach. When the acidified food leaves the stomach and arrives in the duodenum, sodium bicarbonate is secreted to neutralize the acidic gastric juice. Thus, such substances may also be readily applied in the methods according to the invention, as will be appreciated by a person skilled in the art.

During in vivo digestion of a food, a food is typically kneaded throughout the whole gastrointestinal tract. This kneading starts in the oral cavity, where pressure is usually applied to the food by chewing. The purpose of chewing a food is to physically break it into smaller pieces and disrupt its structure. During chewing the food is also mixed with salivary fluid that contains digestive enzymes such as amylase. When the food has been sufficiently chewed, the food is swallowed and enters the oesophagus. The food is now in an “as eaten” state.

It will be clear to a skilled person that when using a method according to the invention to digest a food, it is preferred that the food is or becomes in its “as eaten” state. Not only may this require that the food is processed before digestion, e.g. by cooking it as specified, but also that the food is physically disrupted by a process that mimics chewing. Therefore, in another aspect the invention provides a method for predicting the in vivo glycaemic response to carbohydrates in a food wherein the food is digested in vitro in more than one compartment, comprising applying pressure to said food. Such pressure may be applied by any suitable means. For example, a food may be broken or divided into small pieces by hand force. Also, a food may be ground with a mortar or a food processor.

As mentioned above, the conditions under which the pressure is applied should resemble physiological conditions wherever possible. For example, when grinding a food with a mortar or food processor care should be taken to prevent the temperatures of both the food and the mortar or food processor from deviating largely from body temperature. A skilled person will also appreciate that the intensity and duration of applying pressure to a food to obtain its “as eaten” state depend on the type of the food. For example, less pressure may be required when digesting a semi-solid or non-solid food. It may even be sufficient to not apply any pressure at all, for example when a liquid food such as lemonade or an energy drink is digested. When digesting a solid food such as pumpernickel bread or a lollipop, more pressure will be required.

When the food in vivo has been swallowed and has entered the oesophagus, peristalsis enables further transportation of the food from one digestive compartment to another. Also, peristalsis enhances mixing of the food with the digestive enzymes to improve digestion. Thus, to resemble in vivo conditions pressure is preferably applied in vitro to a food in a peristaltic manner. Under in vitro conditions, peristalsis may be mimicked by mechanical force. Therefore, a method according to the invention may comprise applying pressure to a food, wherein said pressure is applied in a peristaltic manner and wherein said pressure is mechanically induced.

Suitable means for mechanically inducing pressure to a food, preferably in a peristaltic manner, will be known by a person skilled in the art. Said pressure may be induced by pressure chambers that compress a gas or a liquid surrounding a compartment. For example, U.S. Pat. No. 5,525,305 and EP 0 642 382 B1 describe an in vitro model of a gastrointestinal tract comprising a reactor system. This reactor system comprises at least one unit consisting of two or more pressure chambers. A flexible hose is situated in each pressure chamber. This hose is fixed in such a way that a gas or a liquid can either be discharged or exhausted from the spaces between the wall of the pressure chamber and the hose. The discharged gas or liquid induces pressure to the hose, thus inducing pressure to the contents of the hose such as a food. This pressure can be decreased again by exhausting the gas or liquid. The amount of gas or liquid that is discharged or exhausted is controlled by a control means. In this way a pressure can be applied that resembles peristalsis. Thus, the reactor system provides a suitable in vitro method for kneading a food. Furthermore, when using more than one unit multiple pressure chambers can be put into action. By connecting these pressure chambers in a sequential manner also a suitable in vitro method for transporting a food is provided. Thus, a reactor system as described in U.S. Pat. No. 5,525,305 and EP 0 642 382 B1 is very suitable for use in the in vitro methods according to the invention.

It will be appreciated by a person skilled in the art that the method according to the invention can be used to simulate any type of digestive tract, especially when means for applying peristaltic pressure are applied and the compartments are connected to each other in a sequential manner so that a food can be transferred from one compartment to another in a peristaltic motion. Therefore, in another embodiment the method according to the invention comprises transferring a food from one compartment to another in a peristaltic motion. For example, a food is digested with amylase in one compartment, then transferred in a peristaltic motion to another compartment wherein the food is further digested with gastric acid, then transferred in a peristaltic motion to another compartment wherein the food is even further digested with pancreatin, lipase and bile.

Under in vivo conditions the duration of transferring a food from one compartment to another depends on the type of food that is digested. For example, in vivo it takes about 30 minutes to transfer half of the contents of the stomach to the duodenum when a liquid food is digested, and about 40 minutes if a solid food is digested. Therefore, in one embodiment the in vitro method according to the invention comprises transferring a food in a peristaltic motion from a first compartment to a second compartment, according to a controlled transfer curve wherein half of the contents of said first compartment have been transferred to said second compartment in 10 to 60 minutes, preferably in 25 to 45 minutes. For example, a solid food is digested with amylase in a first compartment, gradually transferred in a peristaltic motion from the first compartment to a second compartment, digested with gastric acid and pepsin in the second compartment and then gradually transferred in a peristaltic motion to a third compartment, with a transfer time wherein half of the contents of the second compartment have been transferred to the third compartment in 40 minutes.

It will be clear to a skilled person that the peristaltic motion is ongoing. Thus, when half the contents of a compartment have been transferred by peristaltic motion from that compartment to another compartment, the peristaltic motion continues at least until almost all of the remaining part of the contents also has been transferred to the other compartment. The passage from one compartment to the following compartment can not only be set according to a linear line, but also to an algorithmic curve, for example to simulate realistically the emptying of food from the stomach of humans or a specific animal species related to the type of food.

Also the duration of the in vivo digestion of a food in the intestinal tract depends on the type of food that is digested. Typically, in humans it takes between 2 and 6 hours for a food to pass through the small intestines and to enter the colon. Therefore, in one embodiment the method according to the invention comprises transferring a food from one compartment to a further compartment in a peristaltic motion, wherein the duration of digesting said food in said further compartment is between 60 and 360 minutes, preferably between 110 and 270 minutes. For example, a food is digested with amylase in a first compartment, transferred in a peristaltic motion to a second compartment, digested therein with pepsin, then transferred in a peristaltic motion to a third compartment wherein half the contents of the second compartment have been transferred to the third compartment in 40 minutes, and then digested therein with lipase and pancreatin during 180 minutes.

In order to calculate the in vitro glycaemic response to carbohydrates in a food, at least one sample needs to be collected from that food. Therefore, in yet another aspect the invention provides a method for predicting the in vivo glycaemic response to carbohydrates in a food wherein the food is digested in vitro, characterized in that the food is digested in more than one compartment, comprising collecting at least one sample from said food during and/or after digestion.

As mentioned above, in the art a food's GI is calculated based on the in vivo blood glucose response to the food. The in vivo blood glucose response is typically measured over a total period of 120 minutes, and is characterized by the presence or absence of a peak in the blood glucose level. Both the presence and shape of the peak depend on the type of carbohydrates in the food. For example, simple carbohydrates such as glucose are rapidly digested and absorbed, inducing a fast and relatively large increase in blood glucose values. The resulting blood glucose response curve has a typical shape, characterized by a rapidly occurring, high peak that has a sharp form such as a “spike”. If a food contains more complex carbohydrates that need more digestion time, a more gradual increase in blood glucose levels will be observed. The blood glucose response curve will then display a rise that is much more gradual over time, sometimes without an obvious observable peak.

Thus, it will be appreciated by a person skilled in the art that preferably a number of samples is taken from the in vitro digested food such that an in vitro blood glucose response curve can be obtained that adequately mirrors its in vivo blood glucose response. For example, samples are collected at regular 10 minute intervals during in vitro digestion of a food. The schedule of sampling may also be of a more irregular nature. Samples may for instance be taken every 5 minutes during the first hour of in vitro digestion, and every 15 minutes during the second hour of in vitro digestion.

A person skilled in the art will also appreciate that proper procedures for collecting food samples in vitro must be applied, to avoid sampling errors. For example, aliquots of the digested food may be taken and filtered or dialyzed to remove any solid matter or sediment before collecting a sample from each aliquot. Alternatively, dialysates or filtrates of the food may be obtained at more than one time point during in vitro digestion of the food, and a sample may be collected from each obtained dialysate or filtrate. It will be clear that any in vitro sampling procedure should be standardized for all food samples to be collected in order to obtain representative fractions of the digested carbohydrates.

The final step of in vivo digestion of dietary carbohydrates into monosaccharides such as glucose and fructose is conducted by brush border enzymes. When this final digestion step is completed, the resulting monosaccharides are transported into the epithelial cells. From there, the monosaccharides enter the bloodstream and are transported to the liver for further processing. Thus, a person skilled in the art will appreciate that the carbohydrates in samples collected from an in vitro digested food also need further digestion. Therefore, in one embodiment the method according to the invention comprises further in vitro digestion of at least one carbohydrate in at least one sample collected from an in vitro digested food.

Such further in vitro digestion preferably mimics the in vivo digestion by the brush border enzymes. Thus, the further in vitro digestion is preferably performed by incubating the food samples collected from the earlier in vitro digestion step with brush border enzymes. For example, a food is digested in vitro with amylase in one compartment, transferred in a peristaltic motion to another compartment, digested therein with gastric acid, then transferred to a further compartment wherein food samples are collected at regular 10 minute intervals, and where after the collected food samples are incubated in vitro with brush border enzymes.

It is preferred that the further in vitro digestion is performed under conditions that resemble in vivo conditions. For example, under in vivo conditions it takes some time for the brush border enzymes to further digest the carbohydrates into monosaccharides, depending on the amount and type of carbohydrates in said food. The further in vitro digesting is preferably conducted during a period of between 2 and 24 hours, preferably between 16 and 20 hours (overnight). For example, samples that have been collected from an in vitro digested food are further digested in vitro by incubation with brush border enzymes for 20 hours.

During further in vitro digestion, additional compounds may be added to the collected samples. For instance, one or more compounds may be added that prevent potential microbial growth. Such growth would deplete the glucose concentration in the samples. Any method known in the art can be used to prevent microbial growth in the collected samples, and can be applied by a person skilled in the art. For example, collected samples may be preserved with sodium azide. Also, a skilled person will appreciate the amount of azide to be preventively added. The amount of sodium azide added to the sample may be between 0.01% w/v and 1% w/v, preferably about 0.05% w/v. To prevent microbial growth, also ultrafiltration of the sample may be used.

Although somewhat less preferable, further in vitro digestion of the carbohydrates in a collected food sample may also be conducted by non-enzymatic digestion methods. Such methods are known by a person skilled in the art. For example, it is well known in the art that carbohydrates such as sucrose, maltose and glycogen can be hydrolyzed into their monosaccharides glucose and fructose by acid treatment. Thus, acids are suitable compounds when conducting non-enzymatic hydrolysis of carbohydrates. Therefore, in yet another embodiment the method comprises hydrolyzing at least one carbohydrate in at least one sample collected from a food by an acid. Examples of suitable acids include, but are not limited to, nitric acid, phosphoric acid, sulphuric acid and hydrochloric acid.

As a skilled person will understand, the monosaccharide contents in samples collected from a food may be measured by any means of quantitative analysis that is suitable to measure monosaccharides. For example, the monosaccharide contents can be measured using high performance liquid chromatography (HPLC), high performance ion chromatography (HPIC) or enzymatic analysis, for example a conventional glucose assay or a glucose-fructose assay. These examples of suitable methods for quantitative analysis are not meant to be limitative.

A skilled person will also understand that when using such methods the amount of any monosaccharide can be measured that provides data for determining the in vitro glucose response to a food. For example, the amounts of glucose, fructose and galactose can be measured in the samples collected from a food that was digested in vitro according to the methods of the invention. The measured amounts represent the level of (mono)saccharides at each sampled time point, and can be used to plot the in vitro glucose response curve to that food. The in vivo glycaemic response or GI of the food can then be predicted by comparing the AUC of the in vitro glucose response curve to the AUC of the in vivo glucose response of a reference food.

Therefore, in one embodiment the method comprises measuring the amount of glucose, whether or not in combination with fructose and galactose, in at least one sample collected from a food. For example, a food is digested in vitro with gastric acid in one compartment, transferred in a peristaltic motion to another compartment and then digested therein with digestive enzymes. During the enzyme digestion of the food, samples are collected at regular 5 minute intervals. These samples are then further digested in vitro for 12 hours with brush border enzymes. Finally, the amounts of glucose and fructose in the further digested food samples are determined by for instance a conventional assay.

Once the monosaccharide contents of the samples collected from a food have been measured the in vitro glucose response to the food can be calculated, as mentioned above. Thus, in a further embodiment the method comprises calculating the in vitro glucose response to said food.

Based on the in vitro glucose and fructose response as result of the in vitro digestion steps mentioned above, the in vivo glycaemic response can be predicted. The in vitro glucose and fructose response curves represent the amounts of glucose and fructose that are digested and available for absorption from the intestine to enter the body, In healthy persons the intestinal absorption of glucose and partly of fructose gives an instant reaction in the release of insulin by the pancreas into the blood. This insulin is responsible for lowering the blood glucose concentration after intestinal absorption of glucose and fructose. This means that for the prediction of in vivo blood glucose levels (glycaemic response), based on measured in vitro glucose and fructose response curves, the physiological processes in the body should be taken into consideration.

For this purpose, a kinetic (in silico) model describing these physiological processes involved in glucose homeostasis is used to predict the in vivo glycaemic response to a food based on the obtained in vitro glucose response to that food. This kinetic model is based on the homeostatic model assessment (HOMA) of Wallace et al. (Diabetes Care 2004 vol. 27(6): p. 1487-1495) which can be used to assess β-cell functioning and insulin resistance from baseline (fasting) glucose and insulin concentrations. The HOMA model is mainly based on experimental data collected in human studies and is suitable to predict glucose and insulin concentrations of both diabetic and non-diabetic individuals. This in silico model enables a translation of experimental in vitro glucose data to in vivo plasma glucose profiles. In addition, a glycaemic response that would occur in vivo after ingesting of a certain food product can be predicted.

As mentioned above, foods with a low GI (i.e., foods that elicit a small glycaemic response) are considered to provide significant health benefits in general, especially for subjects suffering from or having increased risk for developing syndromes such as glucose intolerance, insulin resistance, diabetes mellitus, syndrome X or metabolic syndrome, cardiovascular disease and obesity. Foods with a high GI (i.e., foods that elicit a large glycaemic response) may also provide benefits. For example, they are suitable for a person in need of energy recovery after endurance exercise or for a person suffering from diabetes mellitus who is experiencing hypoglycemia.

In a further aspect, the invention therefore provides the use of any method according to the invention for determining the suitability of a food for a subject who would benefit from a normalization of blood glucose levels. In a preferred embodiment, said subject is suffering from or having increased risk for developing at least one syndrome selected from the group of glucose intolerance, insulin resistance, diabetes mellitus, syndrome X, cardiovascular disease and obesity.

The invention is exemplified by the following examples. These examples are intended to illustrate the invention, without limiting the scope thereof in any way.

### EXAMPLE 1

This example describes the in vitro digestion of different types of foods by a method according to the invention. Also the calibration of the in vitro method is described. Furthermore, the accurate prediction of in vivo glycaemic index (GI) values for these foods is demonstrated by comparison of these predicted values to known GI values of reference products.

### In Vitro Digestion of Test Foods

Four different test foods (2 liquid foods, 2 solid foods) were digested in vitro according to the invention As shown in Table 1, for each test food the amount of available carbohydrates (CHO) was standardized.

TABLE 1
Overview of four different foods tested in the in vitro method

| | | Intake of test | Amount of |
|---|---|---|---|
| | | food in in vitro | available CHO |
| Test food | Manufacturer | model | in vivo* |
| 1. Pure glucose | Sigma | 6.25 g | 6.25 g |
| 2. Drink Formula | Nutricia | 35.5 g | 6.25 g |
| (Diasip) | | | |
| 3. White bread | Bolletje | 6.25 g | 6.25 g |
| (Bolletjes Toast) | | | |
| 4. Pumpernickel whole | WEPU Brot | 13.5 g | 6.25 g |
| kernel rye | | | |
| bread | | | |

*Data for amount of available carbohydrates (CHO) are based on the information on the package of the products.

First, each food was processed to an “as eaten” state by processing the food according to instructions provided by its manufacturer and grinding the food into small pieces when necessary. Each food was then individually digested in vitro in duplicate in a two compartment system representing the stomach and small intestine.

For each food the quantity as specified in Table 1 was mixed with artificial saliva, containing 6.2 g/l sodium chloride, 2.2 g/l potassium chloride and 0.3 g/l calcium chloride (for product 1, 57 ml; for product 2, 48 ml; for product 3, 57 ml, for product 4, 44 ml) and drinking water (57 ml; 37 ml; 57 ml; and 63 ml for products 1-4, respectively). The pH of the food-saliva-water mix was set at pH 5.0 and warmed at 30° C.±2° C. The digestion started by the addition of 5.5 mg α-amylase to the food-saliva-water mix and incubation for 1 min. The food-saliva-water-mixture was then immediately introduced into the gastric compartment (pre-warmed to 37° C.±2° C.) of a Tiny-TIM system (TNO gastro-Intestinal Model, TNO, the Netherlands), containing 5 ml gastric juice at pH 2. The total volume in the stomach compartment at the start was 125 ml. During the digestion experiment in the Tiny-TIM system, gastric acid (Merck Hydrochloric acid, 1Mol/L) was added at 0.25 ml/min to the stomach compartment, alternating with water, to control the pH curve in the stomach. Further gastric juice was added at 0.25 ml/min, containing electrolytes, gastric lipase (18 units/ml Rhizopus lipase F-AP 15, Amano Pharmaceuticals) and, pepsin (300 units/ml Sigma P7012). During digestion, the food mix was transferred from the gastric compartment to a small-intestinal compartment in a peristaltic motion. The half time of gastric emptying was set at 40 minutes for solid products and at 30 minutes for liquid products. In the small-intestinal compartment the pH was adjusted to 7.0±0.2 by the addition of sodium-bicarbonate. Further, pig bile (0.25ml/min), pancreatic juice (0.125 ml/min) and small-intestinal electrolytes (0.125 ml/min) were added to this compartment. The available carbohydrates were then absorbed from the food mix through a membrane unit, kept at 37° C., that was connected to the small-intestinal compartment. From the resulting dialysis stream, 1.8 ml samples were continuously collected in duplicate by a fraction collector (Gilson 203B, Gilson Inc., Middleton, USA), at 10 minute intervals for 180 minutes. The collected samples were stored in duplicate 96 deep-well plates. One plate was used for direct analysis. The duplicate plate was sealed and stored at −20° C. or lower.

Plated samples, as well as reference solutions of maltose and sucrose, were further digested in vitro by incubation with brush border enzymes. To this aim, samples and reference solutions were diluted with purified water to concentrations suitable for efficient further digestion. Than, a 1.0 M citrate buffer at pH 6.0 (50 μl) and a brush border enzyme mix were added to each sample and reference solution. The brush border enzyme mix (100 μL) was freshly prepared from Sigma intestinal acetone powders according to instructions (Sigma charge 024K7005) and the addition of lactase (F Amano-50; 1.0 mg/ml). In order to eliminate microbial growth the enzyme solution was ultra-filtered over 0.2 μm cellulose acetate. Digestion of the different monosaccharide components in the samples was carried out at 37° C. over night (20 hours).

### Calibration of the Predictive Model and Prediction of in Vivo Glycaemic Responses and GI Values

Aliquots (0.25 ml; diluted sample with water to an expected glucose concentration not exceeding 0.15% w/v or 1.5 mg/ml) of the digested samples and reference solutions and aliquots of glucose and fructose standard solutions were consequently transferred into micro-titer plates for analysis, using an enzymatic glucose-fructose assay (R-Biopharm AG, Darmstadt, Germany) according to instructions. The concentrations of D-glucose and D-fructose were determined at 340 nm. Obtained glucose and fructose concentrations were multiplied with the dialyzed volume of the Tiny-TIM in vitro system per 10 min. interval to obtain the absolute amount of glucose and fructose per 10 min sampling period.

The obtained in vitro glucose and fructose data were extrapolated to predicted in vivo plasma glucose level profiles by using an in silico model which is based on the HOMA model of Wallace et al. (Diabetes Care 2004 vol. 27(6): p. 1487-1495). This in silico model (Version 2.7) was described using Microsoft® Visual Basic® Macro integrated in Microsoft® Excel® spreadsheets. Model performance was assessed by calculating the average relative deviation of the predicted and in vivo observed glycaemic response curve from 0 to 180 minutes:

{Σ|0-180|((|CGluc Pred−Cgluc Obs|)/CGluc Obs)}/n

The in silico model was calibrated using a dataset from Juntunen et al. (Am. J. Clin. Nutr. 2002 vol. 75: p. 254-262). In that study, a serving of whole kernel rye bread (pumpernickel) with an available carbohydrate load of 50 grams was consumed by young volunteers.

Importantly, a time shift of 10 minutes was used to account for the difference between Tiny-TIM and the onset of glucose absorption in humans. A number of parameters of the in silico model were fitted using constraints on between-organ glucose fluxes in the model and the observed glucose and insulin plasma levels. The final in silico parameters as used in the final version of the in silico model are shown in Table 2. Using these values, the glycaemic response of the model was set to reflect the response of the human volunteer population that participated in the Juntunen study, with an average age of 30 years and a Body Mass Index (BMI) of around 20 kg/m².

TABLE 2
Final calibrated settings for the HOMA in silico model, version 2.7.

| HOMA parameters for young adults | |
|---|---|
| (30 years of age) | |
| VD glucose (I) | 14.00 |
| VD insulin (I) | 13.00 |
| Fasting glucose level (mmol/l) | 5.03 |
| Baseline insulin level (pmol/l) | 45 |
| Insulin degradation (KINS) | 0.17 |
| Liver sensitivity for glucose (LA1) | −2.50 |
| Liver sensitivity for insulin (LA2) | −2.20 |
| Beta Vₘₐₓ | 900.00 |
| Beta IC₅₀ | 7.50 |
| Beta A1 | 5.00 |
| MVMAX | 2.60 |
| MKM | 0.01 |
| MIC₅₀ | 80.00 |
| MA1 | 1.50 |
| MA2 | 2.00 |
| labase | 6.7 |

The glucose and fructose concentration curves in the in vitro dialysates of the test foods, as measured by enzymatic assay, were used as input in the in silico model to predict the human in vivo plasma glucose and insulin responses in healthy subjects. For the in vitro fructose concentration it was calculated that it contributed to the glycaemic response for 20% in comparison to the in vitro glucose concentration. To this aim, the predicted in vivo blood glucose curves were compared to actual in vivo glycaemic response curves observed in humans after intake of similar products (Lee et al., Eur J Clin Nutr 1998 vol. 52: 924-928, Hofman et al., Eur J Clin Nutr 2004 vol. 58: 1553-1556, Hofman et al., Asia Pac J Clin Nutr 2006 vol. 15(3): 412-417, Juntunen et al., Am J Clin Nutr 2002 vol. 75: 254-262, Foster-Powell et al., Am J CLin Nutr 2002 vol. 76(1): 5-56 and Liljeberg et al., Eur J Clin Nutr 1992 vol. 46(8): 561-575).

When data from literature were given as plasma glucose concentration corrected for baseline, the data were adjusted back using a fasted plasma glucose concentration of 5.03 mmol/l (Genuth et al., Diabetes Care 2003 vol. 26: 3160-3167). Also a preliminary similarity criterion was used. Similarity was considered when the peak of the obtained in vitro glycaemic curve deviated no more than 10% of its maximum value and not more than 10-15 minutes in appearance from the in vivo curve. Finally, also the GI of the test foods was calculated, based on the AUC for the predicted plasma glucose curves.

### Results and Conclusion

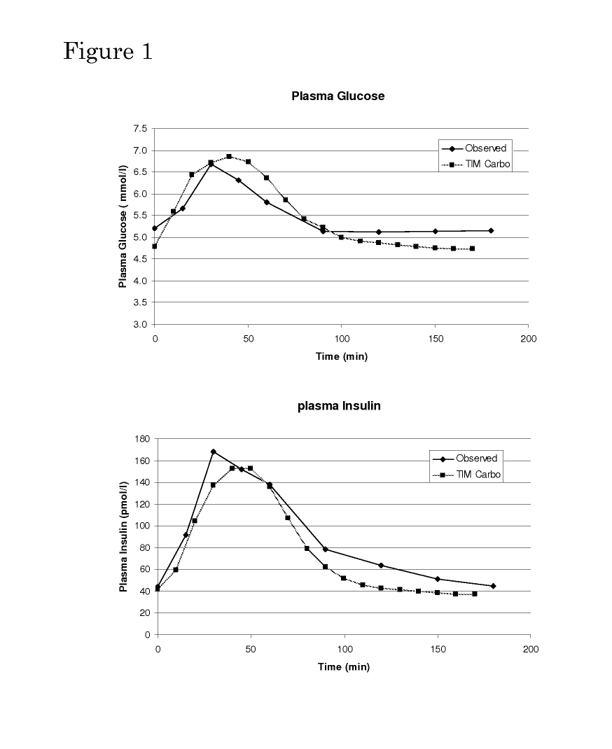

FIG. 1 shows the prediction by the in silico model of the in vivo glucose and insulin responses to a 50 grams pumpernickel meal in young volunteers, based on the calibration data of the Juntunen study. Analysis revealed that the predicted glucose response deviated no more than 6.7% from the actual in vivo response observed by Juntunen et al.

Table 3 shows the predicted and actual in vivo maximum glucose concentrations (Cₘₐₓ) and the predicted incremental areas under the curve (iAUC) of the test foods. It can be observed that each predicted Cₘₐₓ closely resembles the actual Cₘₐₓ that was observed in vivo. Also shown are the GI values of the test foods, as calculated by the in silico model based on their iAUC. The GI of pure glucose was calculated at 100, the GI of white bread at 74.

Thus, as demonstrated by FIG. 1 and Table 3, the in vitro methods according to the invention reliably predict the in vivo GI and glycaemic response to both solid and non-solid foods.

TABLE 3
Predicted and actual in vivo Cₘₐₓ and predicted iAUC of the test foods.
Also shown are the calculated GI values of the test foods, based on their
predicted iAUC and related to white bread and glucose as reference foods.

| | Predicted | Actual | | GI (white | GI |
|---|---|---|---|---|---|
| | Cₘₐₓ | Cₘₐₓ | Predicted | bread | (glucose |
| Test food | (mmol/l) | (mmol/l) | iAUC | as ref.) | as ref.) |
| Pure glucose | 8.35 | 8.62 | 127.91 | 136 | 100 |
| Drink formula | 5.68 | 5.48 | 9.36 | 10 | 7 |
| White bread | 6.9 | 6.9 | 94.16 | 100 | 74 |
| Pumpernickel | 6.9 | 6.7 | 95.51 | 101 | 75 |

### EXAMPLE 2

This example describes a further experiment, wherein in vivo glycaemic responses were predicted for multiple solid and non-solid test foods based on the in vitro method according to Example 1. For each test food, the predicted in vivo response was again compared to its actual in vivo glycaemic response as observed in human study populations. The actual in vivo responses were provided by the manufacturers of the test foods, together with demographical data describing the human study populations.

Table 4 lists the different food products that were tested in this experiment. For each type of test food the amount of available carbohydrates (CHO) was standardized.

TABLE 4
Overview of foods tested in the in vitro method. CHO = carbohydrates,
HM = High Maize

| | | Intake of | Amount |
|---|---|---|---|
| | | test food | of available |
| | | in in vitro | CHO |
| Test food | Manufacturer | model | in vivo* |
| Durum wheat semolina pasta | Barilla | 9.3 g | 50.0 g |
| (Spaghetti Integrali) | | | |
| Parboiled pearled wheat | Barilla | 9.61 g | 50.0 g |
| Legume pasta (Spaghetti Plus) | Barilla | 10.33 g | 50.0 g |
| Soft bread | Barilla | 13.24 g | 50.0 g |
| High-fibre biscuit | Barilla | 11.04 g | 50.0 g |
| Breakfast Rusk | Barilla | 8.23 g | 50.0 g |
| Nutrient formula A | Abbott | 49.45 g | 25.0 g |
| Nutrient formula B | Abbott | 39.66 g | 50.0 g |
| Breakfast cereals (All Bran) | Kellogg | 14.9 g | |
| Breakfast cereals (Special K) | Kellogg | 7.6 g | |
| soluble fibre A | Tate & Lyle | 3.13 g | 25 g |
| soluble fibre B | Tate & Lyle | 3.13 g | 25 g |
| soluble fibre E | Tate & Lyle | 3.13 g | 25 g |
| insoluble fibre C | Tate & Lyle | 3.13 g | 25 g |
| insoluble fibre D | Tate & Lyle | 3.13 g | 25 g |
| Control bread | Nat. Starch | 8.9 g | 36 g |
| HM260 bread dose 1 | Nat. Starch | 9.0 g | 29.4 g |
| HM260 bread dose 2 | Nat. Starch | 9.1 g | 28.0 g |

*Data for amounts of available carbohydrates (CHO) were provided by the food's manufacturers.

Table 5 displays the demographical data of the human study populations for each tested food, including gender, age and body mass index (BMI). The number of study subjects ranged from 6 to 22 subjects per population.

TABLE 5
Demographical data of the human study groups. BMI = Body Mass Index
(kg/m²), HM = High Maize, m = male, f = female.

| | Number | | | |
|---|---|---|---|---|
| | of study | | | |
| Food | subjects | Gender | Age (years) | BMI (kg/m²) |
| Durum wheat | 10 | 3 female, | 26.5 ± 2.5* | 21.9 ± 0.9* |
| semolina pasta | | 7 male | | |
| (Spaghetti Integrali) | | | | |
| Parboiled pearled | 9 | 7 female, | 26.2 ± 1.8* | 21.6 ± 0.7* |
| wheat | | 6 male | | |
| Legume pasta | 10 | 3 female, | 26.5 ± 2.5* | 21.9 ± 0.9* |
| (Spaghetti Plus) | | 7 male | | |
| Soft bread | 10 | 3 female, | 26.5 ± 2.5* | 21.9 ± 0.9* |
| | | 7 male | | |
| High-fibre biscuit | 10 | 3 female, | 25.2 ± 0.9* | 23.4 ± 1.1* |
| | | 7 male | | |
| Breakfast Rusk | 10 | 5 female, | 25.7 ± 2.6* | 21.9 ± 0.6* |
| | | 5 male | | |
| Nutrient formula A | 10 | 4 female, | 20.2-38.7 | |
| | | 6 male | | |
| Nutrient formula B | 10 | 3 female, | 21.6-28.6 | |
| | | 7 male | | |
| Breakfast cereals | 6 | male | 27.8 ± 1.5* | 22.8 ± 0.24* |
| (All Bran) | | | | |
| Breakfast cereals | 6 | male | 27.8 ± 1.5* | 22.8 ± 0.24* |
| (Special K) | | | | |
| soluble fibre A | 22 | mixed | 25-45 | 20-28 |
| soluble fibre B | 12 | mixed | 26-36 | 23-27 |
| soluble fibre E | 12 | mixed | 26-36 | 23-27 |
| insoluble fibre C | 12 | mixed | 26-36 | 23-27 |
| insoluble fibre D | 12 | mixed | 26-36 | 23-27 |
| Control bread | 20 | 12 m + 8 f | 27.5 ± 17 | 24.3 ± 1.0 |
| HM260 bread dose 1 | 20 | 12 m + 8 f | 27.5 ± 17 | 24.3 ± 1.0 |
| HM260 bread dose 2 | 20 | 12 m + 8 f | 27.5 ± 17 | 24.3 ± 1.0 |

*mean ± Standard Error of the Mean (SEM)

### In Vitro Digestion of Test Foods

Each food was digested in vitro exactly according to the method as described in Example 1, with one minor adaptation. After all samples had been collected from the dialysates, 0.05% w/v sodium azide was added to the samples and reference solutions just before the overnight incubation with the brush border enzymes. The sodium azide was added to prevent any possible microbial growth.

### Prediction of in Vivo Glycaemic Responses

The glucose and fructose concentrations of each sample were exactly measured as described in Example 1.

As for the in silico model, the baseline plasma glucose and insulin values were adapted to the respective values as provided by the manufacturers of the tested foods. The adapted baseline plasma glucose concentrations ranged from 3.97 mmol/l to 5.19 mmol/l. The adapted baseline insulin concentrations ranged from 29.6 pmol/l to 45.6 pmol/l. The in vivo glycaemic responses to the test foods were then predicted exactly as described in Example 1. No GI values were calculated this time.

The experiment provided 15 predicted plasma glucose response curves, one for each tested food. These predicted curves were then compared to the actual in vivo plasma glucose response curves. The maximum glucose concentrations (Cₘₐₓ) of both the predicted and actual in vivo plasma glucose response were compared with each other, with regard to the time of appearance and the absolute glucose value. Also the average deviation of the predicted integrated area under the curve (iAUC) from the actual in vivo AUC was calculated, per measured time interval.

Results and conclusions

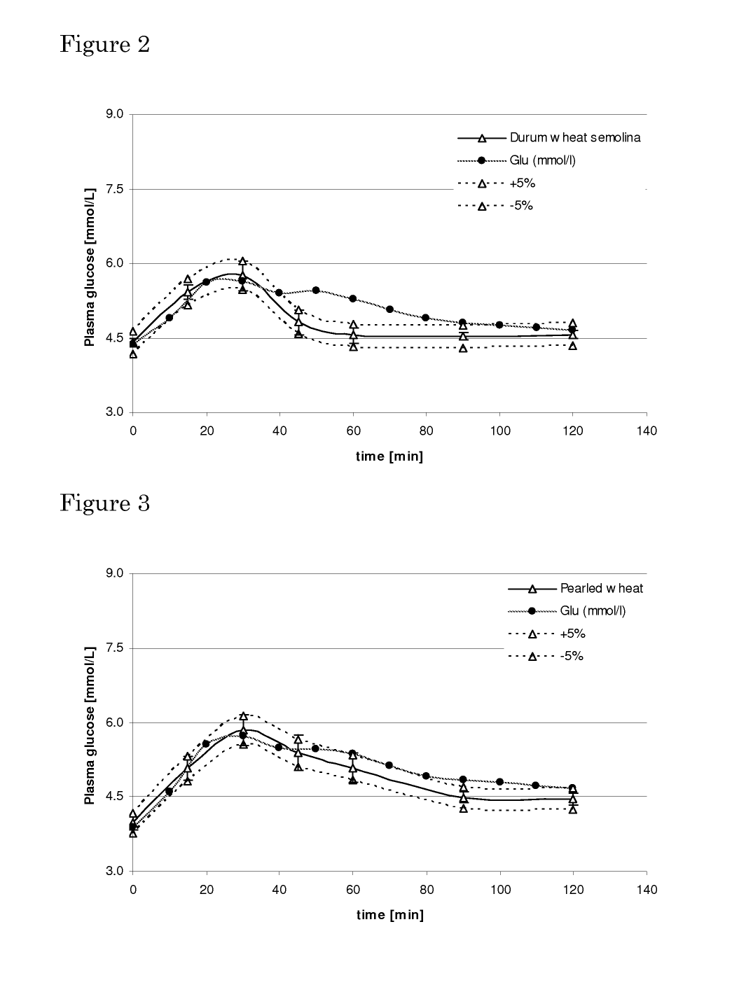

FIGS. 2 to 5 display some of the predicted plasma glucose response curves. In all figures, both the glucose response curve as predicted by the method according to the invention and the average actual in vivo glucose response curve are shown. In addition, lines are added indicating±5% deviation from the actual in vivo glucose response curve.

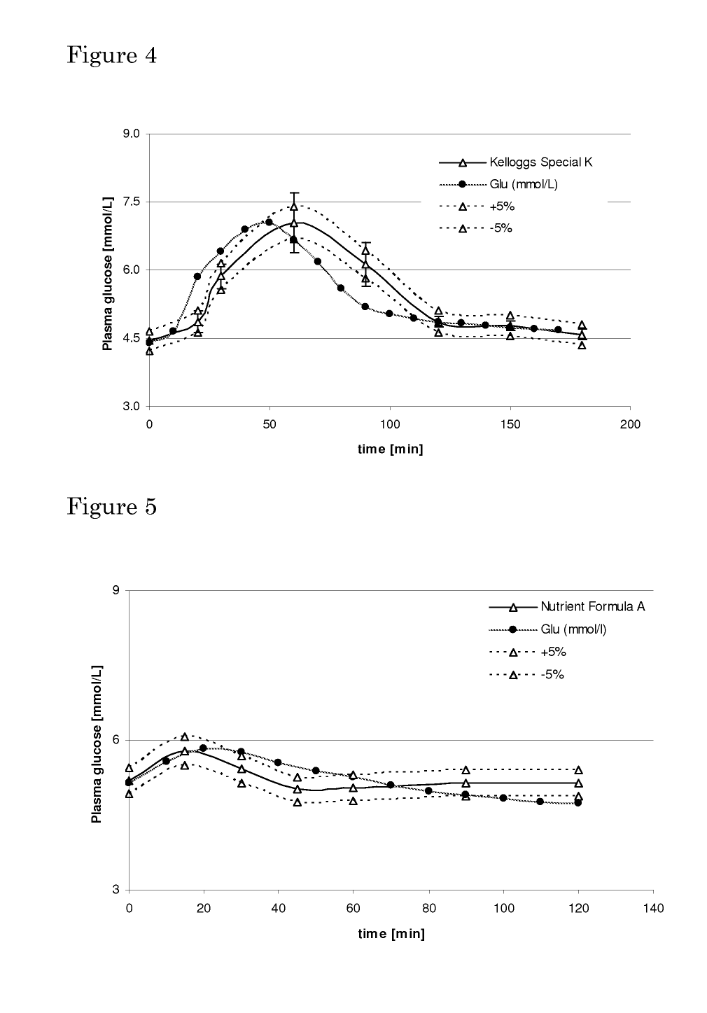

It can be observed from FIG. 2 (durum wheat semolina pasta) that both the predicted and actual in vivo maximum glucose concentrations (Cₘₐₓ) occur at the same time and are of equal magnitude. A similar result can be observed in FIG. 3 (parboiled pearled wheat), wherein the predicted glucose response curve closely matches and follows the actual in vivo glucose response curve. FIGS. 4 (Special K breakfast cereals) and 5 (Nutrient Formula A) show a predicted glucose response curve wherein Cₘₐₓ occurs somewhat earlier (FIG. 4) or later (FIG. 5) than the actual in vivo Cₘₐₓ. In both figures, the predicted glucose response curve again has a similar shape as the actual in vivo glucose response curve.

After 50 to 90 min. the predicted glucose response curve is often slightly different from the in vivo glycaemic response. A reason might be, that the first glucose response curve (up to Cₘₐₓ) is mainly driven by the rate of digestion and absorption, while at later time points the blood glucose concentrations will be mainly determined by the rate at which glucose is removed via insulin-dependent and non-insulin dependent mechanisms. (Kendall et al., 2008, Effect of novel maize-based dietary fibres on postprandial glycaemia and insulinemia).

Table 6 shows the results of the comparisons of the predicted Cₘₐₓ glucose values and iAUC with the actual in vivo Cₘₐₓ and AUC. It can be observed that the predicted Cₘₐₓ glucose values closely resemble the actual in vivo Cₘₐₓ values. Larger differences can be observed when comparing the predicted iAUC versus the actual AUC. The average deviation of the predicted iAUC from the actual in vivo AUC, per measured time interval, ranged between 3.6% (parboiled pearled wheat) and 14.4% (soft bread).

TABLE 6
Comparisons of predicted Cₘₐₓ and integrated area under the curve (iAUC, 0-120
minutes) with actual in vivo Cₘₐₓ and AUC. Also shown are the average deviations of the
predicted iAUC from the actual in vivo AUC per measured time interval.

| | | | | | Average |
|---|---|---|---|---|---|
| | | | | | deviation |
| | Predicted | Actual | | | from |
| | Cmax | Cmax | Predicted | | actual |
| Test food | (mmol/l) | (mmol/l) | iAUC | Actual AUC | AUC (%) |
| Durum wheat semolina | 5.64 | 5.75 | 81.9 | 55.2 ± 6.7 | 6.0 |
| pasta (Spaghetti Integrali) | | | | | |
| Parboiled pearled | 5.74 | 5.85 | 133.3 | 114.5 ± 19.3 | 3.6 |
| wheat | | | | | |
| Legume pasta | 5.71 | 5.53 | 103.9 | 55.8 ± 12.6 | 8.4 |
| (Spaghetti Plus) | | | | | |
| Soft bread | 7.09 | 6.31 | 185.9 | 92.1 ± 9.2 | 14.4 |
| High-fibre biscuit | 5.67 | 5.65 | 95.77 | 59.7 ± 7.7 | 6.3 |
| Breakfast Rusk | 6.25 | 6.43 | 132.9 | 95.5 ± 19.5 | 6.9 |
| Nutrient formula A | 5.83 | 5.79 | 53.5 | — | 5.0 |
| Nutrient formula B | 7.08 | 7.05 | 99.7 | — | 6.0 |
| Breakfast cereals | 4.96 | 5.65 | 57.3 | — | 4.8 |
| (All Bran) | | | | | |
| Breakfast cereals | 7.05 | 7.04 | 180.63 | — | 7.4 |
| (Special K) | | | | | |
| Soluble fibre A | 4.9 | 5.56 | 29.74 | — | 7.0 |
| Soluble fibre B | 4.78 | 5.16 | 44.3 | — | 5.3 |
| Soluble fibre E | 5.15 | 5.92 | 33.4 | — | 7.6 |
| Insoluble fibre C | 4.79 | 4.53 | 41.6 | — | 6.3 |
| Insoluble fibre D | 4.78 | 4.72 | 25.6 | — | 5.3 |
| Control bread | 6.25 | 6.24 | 125 | 93.6 ± 12.5 | 9.1 |
| HM260 bread dose 1 | 5.9 | 6.16 | 91.3 | 65.1 ± 7.8 | 10.7 |
| HM260 bread dose 2 | 5.78 | 6.06 | 86.5 | 61.5 ± 8.2 | 11.0 |

“—” means data not available.

Cₘₐₓ of durum wheat semolina pasta, parboiled wheat, legume pasta, high fibre biscuit, breakfast rusk, Kellogg's All Bran and Insoluble fibre D appeared during the same time interval. Cₘₐₓ of the soft bread, the drink formula A, the Soluble fibre A, B, the insoluble fibre E and C occurred 5 to 10 minutes earlier in vivo as compared to the predicted curves, whereas Cₘₐₓ for the drink formula B was measured 10 minutes later in vivo as compared to the predicted value.

It was mentioned above that the predicted plasma glucose curve is shifted about ten minutes to compensate for the onset of the glucose absorption in vivo and the experiments herein conducted. The predicted plasma glucose curve of Kellogg's Special K demonstrates this effect (FIG. 4). In this experiment, the glucose measurement must have been started already with the ingestion of the test product, rather than directly thereafter, since the in vivo plasma glucose curves increases only after 20 minutes.

The Cₘₐₓ values deviated less than 5% for durum wheat semolina pasta, parboiled pearled wheat, legume pasta, high fibre biscuit, breakfast rusk, Nutrient formula A and B, breakfast cereals (Special K), Soluble fibre B and Insoluble fibre D between the in vivo values and the predicted values. Cₘₐₓ deviated between 5 and 15% for the soft bread, Kellogg's All Bran, Soluble fibre A, -B, -E and -C. Cₘₐₓ of Soluble fibre E deviated the most with 15%, whereas the other products diverged less than 11%.

The deviation per measured time interval of both curves from each other showed a deviation not more than 9%, except the soft bread; where both curves diverged approximately 14% form each other.

For three types of bread, whether or not enriched with Hi Maize 260, the order of predicted plasma glucose curves is exactly equal to what was found in vivo, with the control bread resulting in the highest plasma glucose response curves and thereafter HM 260 dose 1 and subsequently HM 260 dose 2. Even the crossing between both curves of HM 260 dose 1 and HM 260 dose 2 which occurs in vivo after approximately 35 minutes is visible in the predicted curves at approximately t=25 (data not shown). This indicates that the in vitro method according to the invention is able to detect minor differences in true digestibility and to translate this into the appropriate order of predicted plasma glucose response curves.

The first half of the predicted versus the in vivo plasma response curves are similar to each other until Cₘₐₓ is reached. Thereafter, the predicted plasma glucose curves strive towards their steady state level, which was set at 4.63 mmol/L. In contrast, the in vivo glucose response plasma curves decrease below the initial fasting glucose plasma level. This is most probably due to the insulin secretion, which induces a more sudden decrease and a steeper slope of the plasma glucose curves in vivo even below the fasting plasma glucose level.

Some differences between the predicted curves and the plasma glucose levels obtained from the in vivo studies appear rather large. However, in vivo a broad inter individual range as well as very varying fasting plasma glucose baseline values (3.97 mmol/L-5.19 mmol/L) were observed (data not shown). This depends on e.g. the age of the study population, their hunger status and their BMI. The major part of the study populations to which the predicted plasma glucose curves were compared to were young, lean and healthy adults. The in silico model was calibrated to predict plasma glucose levels of this type of population and the method according to the invention yields values that correspond to the average individual.

In conclusion, the tested plasma glucose curves were reliably predicted in vitro with a deviation of not more than 9%. The often very high inter subject variability that occurs in vivo is not observed in the methods according to the invention, as the conditions are highly standardized and reproducible. Therefore the methods according to the invention provide very useful tools to study the glucose response in vitro, and to adequately predict the in vivo glycaemic response to a food.

1. A method for predicting the in vivo glycaemic response to carbohydrates in a food wherein the food is digested in vitro, the food is digested in more than one compartment.

2. Method according to claim 1, wherein said food is digested in two compartments.

3. Method according to claim 1, comprising digesting said food with at least one enzyme selected from the group consisting of amylases, lipases, and proteinases.

4. Method according to claim 1, comprising digesting said food at a specific pH value that may be different between compartments.

5. Method according to claim 1, comprising applying pressure to said food.

6. Method according to claim 5, wherein said pressure is applied in a peristaltic manner.

7. Method according to claim 5, wherein said pressure is mechanically induced.

8. Method according to claim 1, comprising transferring said food from one compartment to another in a peristaltic motion.

9. Method according to claim 8, wherein said peristaltic motion is mechanically induced.

10. Method according to claim 1, comprising collecting at least one sample from said food during and/or after digestion.

11. Method according to claim 10, comprising further in vitro digesting at least one carbohydrate in said at least one sample collected from said food.

12. Method according to claim 10, comprising measuring the amount of at least one monosaccharide.

13. Method according to claim 12, comprising calculating the in vitro glucose response to said food.

14-15. (canceled)

16. Method according to claim 3, wherein the enzyme is selected from the group consisting of pepsin, trypsin, chymotrypsin, pancreatin and brush border enzymes.

17. Method according to claim 12, wherein said monosaccharide is selected from the group consisting of glucose, fructose and galactose in said at least one sample collected from said food.
